(19) United States
(12) Patent Application Publication
(10) Pub. No.: US 2007/0119710 A1
(43) Pub. Date: May 31, 2007

(54) Test substrate handling apparatus

(76) Inventors: Daniel Goldberger, Boulder, CO (US); Eric Shreve, Louisville, CO (US); Wayne Siebrecht, Golden, CO (US); Benny Pesach, Rosh Haayin (IL); Gidi Pesach, Kfar Vitkin (IL); Gabby Bitton, Jerusalem (IL); Ron Nagar, Tel Aviv (IL); Dalia Argaman, Hod-Hasharon (IL); Stephen Bellomo, Zicron Yacov (IL); Robert Larson, Perkasie, PA (US); Larry Johnson, Golden, CO (US); Jill Klomhaus, Niwot, CO (US)

Correspondence Address: PATENTMETRIX, 14252 CULVER DR. BOX 914, IRVINE, CA, 92604, US

(21) Appl. No.: 11/288,033

(22) Filed: Nov. 28, 2005

(51) Int. Cl. G01N 33/487

(52) U.S. Cl. 204/403.01

(57) ABSTRACT

The present invention is an integrated, test substrate handling apparatus for the measurement and analysis of blood analytes and blood parameters. The test substrate handling apparatus includes multiple test substrate components combined in a single unit. It advances a test substrate for measurement of blood parameters via programmed, periodic blood sampling or via operator input.

### FIELD OF THE INVENTION

The present invention relates generally to systems and methods for automatically measuring physiological parameters and, more specifically, to methods and systems for an integrated test substrate handling mechanism. More specifically, the present invention relates to improved methods and systems for storing and positioning a test substrate or plurality thereof used in the measurement of physiological blood parameters. In addition, the present invention relates to methods and systems for storing, positioning, and using test substrate elements in conjunction with an automated blood parameter measurement device, and more specifically to a device for measuring blood glucose levels that employs test strips as the test substrate.

### BACKGROUND OF THE INVENTION

Patient blood chemistry and monitoring of patient blood chemistry are important diagnostic tools in patient care. For example, the measurement of blood analytes and parameters often provide much needed patient information regarding the proper dose and administration time period. Blood analytes and parameters tend to change frequently, however, especially in the case of a patient under continual treatment, thus making the measurement process tedious, frequent, and difficult to manage.

For example, diabetes mellitus can contribute to serious health problems because of the physical complications that can arise from abnormal blood glucose levels. In the United States alone, it is estimated that over 11 million people suffer from diabetes. The two most common forms of diabetes are Type I, juvenile-onset, and Type II, adult-onset. Type I diabetes destroys the vast majority of the insulin-producing beta cells in the pancreas, thus forcing its sufferers to take multiple daily insulin injections. Type II diabetes is usually less severe than Type I, causing a decreased level of endogenous insulin production in the body, and can often be controlled by diet alone.

The body requires insulin for many metabolic processes; it is chiefly important for the metabolism of glucose. If normal blood glucose levels are maintained throughout the day, it is believed that many of the physical complications associated with diabetes could be avoided. Maintaining a consistent and normal blood glucose level is an arduous task as the diabetic's blood glucose level is prone to wide fluctuations, especially around mealtime. Many diabetics are insulin dependent and require routine and frequent injections to maintain proper blood glucose levels.

Unlike the normal functioning of the body's glucose control systems, injections of insulin do not incorporate feedback mechanisms. Controlling glucose levels therefore requires continuous or frequent measurements of blood glucose concentration in order to determine the proper amount and frequency of insulin injections. The ability to accurately measure analytes in the blood, particularly glucose, is important in the management of diseases such as diabetes as described above. Blood glucose levels must be maintained within a narrow range (about 3.5-6.5 mM). Glucose levels lower than this range (hypoglycemia) may lead to mental confusion, coma, or death. High glucose levels (hyperglycemia) cause excessive thirst and frequent urination. Sustained hyperglycemia has been linked to several complications of diabetes, including kidney damage, neural damage, and blindness.

Conventional glucose measurement techniques require lancing of a convenient part of the body (normally a fingertip) with a sharp lancet, milking the finger to produce a drop of blood at the impalement site, and depositing the drop of blood on a measurement device (such as an analysis strip). This lancing method, at typical measurement frequencies of two to four times a day, is both painful and messy for the patient. The pain and inconvenience has additional and more serious implications of noncompliance. Patients generally avoid maintaining the recommended regimen of blood glucose measurement and thereby run the risk of improper glucose levels and consequent harmful effects.

Conditions worsen when there is a need for frequent blood glucose determination, such as when a diabetic patient is acutely ill, undergoing surgery, pregnant (or in childbirth), or suffering from severe ketoacidosis.

Conventional point-of-care (POC) techniques for diagnostic blood testing are routinely performed manually at the bedside using a small sample of blood. In addition, as mentioned above, home glucose monitoring by diabetics is also becoming increasingly routine in diabetes management. Patients are typically required to maintain logbooks for manually recording glucose readings and other relevant information. Even more specifically, patients now measure their blood glucose at scheduled times to determine the amount of insulin needed based on the current blood glucose result, and then record this information in a personal log book.

Operationally, the user first pricks a finger or other body part using a lancing device to produce a small sample of blood. The user then opens a vial of test strips, removes a test strip and inserts the test strip into the meter. The user then must re-close the vial and check for the correct calibration code on the meter. Subsequently, the user transfers the sample to the test strip.

Generally, a user is required to transfer a specific volume of sample to a specific location on the small test strip, which is an added and complicated step for the individual user. After the test sample is deposited onto the test strip, the test strip is inserted into the glucose measurement device. The electrode system on the test strip generates a signal indicative of an amount of glucose in the blood sample, and transmits this signal to the glucose meter, which displays and stores the reading.

The above mentioned individual test strip vial storage mechanism has substantial disadvantages, however. Typical test strips are very short in length—approximately two centimeters. This length is considered to be the minimum practical length, to allow a user to easily handle the test strips. In addition, attempts have been made to shorten the required length of the test strip in other to minimize the resources required to produce a test strip and thus increase test strip production while reducing cost. However, test strips that are shorter than the conventional two centimeters are generally harder to handle, increasing the difficulty of placing and removing the test strip from the meter without dropping the test strip or contaminating the user with blood. Test strip limitations are exacerbated by the fact that diabetics often have poor eyesight and generally have difficulty in handling test strips.

Moreover, current test strips are usually stored loosely in a vial, which results in an inefficient utilization of space. A user must open the vial, reach into the vial and grab and remove one of the test strips from the vial interior. The user must then remember to re-seal the vial; otherwise the test strips will deteriorate due to exposure to humidity and will be rendered unusable. The user must then manually handle the test strip, insert the test strip into the glucose meter, lance the skin, obtain a drop of blood and transfer the blood sample onto the test strip. Finally, after the analysis, the user is required to remove the test strip.

In addition, the residual fluid sample on the glucose meter may interfere with subsequent readings due to the possible contamination of the dispenser valve and lancing device incorporated therein.

In the light of the above-described disadvantages, there is a need for improved methods and systems that can provide comprehensive blood parameter testing. Thus, what is needed are methods and apparatuses for handling chemical strips that are economical, convenient to use, and eliminates the need for the handling of individual chemical strips.

What is also needed is a method and apparatus that is used for the storage of test substrate, the positioning of test substrate for testing, and the storage of contaminated material.

What is also needed is a method and apparatus for automatically handling chemical test substrate strips, thus obviating the needs for individual test strip handling.

In addition, what is needed is a chemical test substrate handling apparatus in which a cleaning pad is incorporated to minimize contamination.

### SUMMARY OF THE INVENTION

The present invention is directed towards an integrated, test substrate handling apparatus for measurement and analysis of blood analytes and blood parameters. The present invention is also directed towards a test substrate handling apparatus in which test substrate components are combined in a single unit that advances a test substrate for measurement of blood parameters via programmed, periodic sampling or via operator input.

In one embodiment, the test substrate handling device of the present invention is employed for storing test substrates or strips and positioning the substrate for subsequent testing. In another embodiment, the test substrate handling device of the present invention is also employed for holding used test substrate elements. In yet another embodiment, the test substrate handling device of the present invention is used for holding residual test fluid.

In another embodiment, the test substrate handling apparatus of the present invention is a strip handling carousel in which chemically-based test strips are held for storage, positioning for subsequent testing, and for holding residual test fluid. The individual test strips are advanced when a testing event is initiated.

In another embodiment, the test substrate handling apparatus is a cassette, wherein the cassette houses new and used test substrates in separate, sealed compartments. The cassette also houses a sample dispenser, which is sealed from both new and used test substrate compartments. Preferably, the cassette comprises a tape compartment seal, which allows for the tape to pass through from area to area, but does not allow air or fluid to flow back into the dispensing area or the new test substrate area. The monitor interface is sealed as part of the cassette, and may be optical, electrical, or any other suitable monitoring means.

In another embodiment, the test substrates are positioned onto a test substrate laminate, which is preferably a continuous tape. Thus, the test substrate is in a position optimal for presenting it to the sample dispenser. The continuous tape is wrapped around a rotary drum on both ends. The test substrates are preferably spaced such that the next new test substrate is sealed in the unused tape compartment and that the used substrate is sealed in the used compartment. The test substrate position allows the host monitor to read the test substrate through the monitor interface.

In another embodiment, the cassette further comprises a cover layer dispenser, which is used to deposit a cover layer onto the tape laminate to encapsulate the used test substrate to reduce possible contamination across seal barriers.

In another embodiment, the test substrate laminate also comprises pads for purging the sample dispenser and cleaning the testing area.

In another embodiment, the test substrate apparatus is used with a blood parameter measurement system in which the system operates automatically to draw blood samples at suitable, programmable frequencies to analyze the drawn blood samples and obtain the desired blood readings such as glucose levels, hematocrit levels, hemoglobin blood oxygen saturation, blood gasses, lactates or any other parameter as would be evident to persons of ordinary skill in the art.

In another embodiment, the present invention comprises an apparatus for holding and positioning a plurality of test substrates for analysis and measurement of blood parameters, comprising: a first storage means for storing unused test substrate; a means for advancing and individually positioning at least one unused test substrate for receiving a sample from a sample dispenser; and a second storage means for storing used test substrate. The apparatus can comprise a cassette and a strip handling carousel. The first storage means and said second storage means is a sealed compartment within the cassette.

In another embodiment, an apparatus for holding and positioning a plurality of test substrates for analysis and measurement of blood parameters, comprising a cassette, wherein said cassette further comprises an outer case; a first sealed compartment for storing a plurality of unused test substrates; a second sealed compartment, for storing a plurality of used test substrates; a test substrate pathway extending between said first sealed compartment and said second sealed compartment a sample dispenser in fluid communication with said pathway; and a monitor interface. The apparatus can further comprise a tape laminate, wherein said tape laminate is used to hold the test substrate. The cassette further comprises a laminate tape compartment seal. The cassette further comprises at least one plastic roller for guiding the test substrate through the cassette. The cassette further comprises a gear for advancing the test substrate through the cassette. The cassette further comprises a tape laminate cover roll.

### BRIEF DESCRIPTION OF THE DRAWINGS

These and other features and advantages of the present invention will be appreciated, as they become better understood by reference to the following detailed description when considered in connection with the accompanying drawings, wherein:

FIG. 1 is a top perspective view of one embodiment of a test substrate handling apparatus of the present invention;

FIG. 2 is an enlarged cross-sectional view of one embodiment of a test substrate handling apparatus of the present invention, as shown in FIG. 1;

FIG. 3 is a detailed illustration of one embodiment of the test substrate handling apparatus of the present invention;

FIGS. 4a, 4b, and 4c each depict one embodiment of the test substrate tape laminate of the present invention;

FIG. 5 is an illustration of one embodiment of an exemplary tape compartment seal of the test substrate handling apparatus of the present invention;

FIG. 6 depicts one embodiment of a cover layer for the test substrate laminate tape employed in the test substrate handling apparatus of the present invention; and

FIGS. 7a, 7b, and 7c illustrate the operational steps of one embodiment of the chemical test substrate handling apparatus further comprising a cleaning pad, used in conjunction with a fluid dispensing system for fluid test samples.

### DETAILED DESCRIPTION OF THE INVENTION

The present invention is directed towards an integrated, test substrate handling apparatus for measurement and analysis of blood analytes and blood parameters. The present invention is also directed towards a test substrate handling apparatus in which test substrate components are combined in a single unit that advances a test substrate for measurement of blood parameters via programmed, periodic blood sampling or via operator input.

In one embodiment, the test substrate handling device of the present invention is employed for storing test substrates or strips and positioning the substrate for subsequent testing. In another embodiment, the test substrate handling device of the present invention is also employed for holding used test substrate elements. In yet another embodiment, the test substrate handling device of the present invention is used for holding residual test fluid.

In another embodiment, the test substrate handling apparatus of the present invention is a strip handling carousel in which chemical test strips are held for storage, positioning for subsequent testing, and for holding residual test fluid. The individual test strips are advanced when a testing event is initiated. The strip handling carousel may or may not be sealed in a cassette or container. Operationally, it is used with an actuator mechanism that rotates the strip handling carousel into a measurement position in relation to a testing apparatus, such as a blood glucose testing apparatus or a specific monitor interface. Once a test is conducted, the actuator mechanism returns the carousel to a resting, non-engaged state.

In another embodiment, the test substrate handling apparatus is a cassette, wherein the cassette houses new and used test substrates in separate, sealed compartments. The cassette also houses a sample dispenser, which is sealed from new and/or used test substrate compartments, or both. Preferably, the cassette comprises a tape compartment seal, which allows for the tape to pass through from area to area, but does not allow air or fluid to flow back into the dispensing area or the new test substrate area. The monitor interface is sealed as part of the cassette, and may be optical, electrical, or any other suitable monitoring means.

In another embodiment, the test substrates are positioned onto a test substrate laminate, which is preferably a continuous tape. Thus, the test substrate is in a position optimal for presenting it to the sample dispenser. The continuous tape is wrapped around a rotary drum on both ends. The test substrates are preferably spaced such that the next new test substrate is sealed in the unused tape compartment and that the used substrate is sealed in the used compartment. The test substrate position allows the host monitor to read the test substrate through the monitor interface.

In another embodiment, the cassette further comprises a cover layer dispenser, which is used to deposit a cover layer onto the tape laminate to encapsulate the used test substrate to reduce possible contamination across seal barriers.

In another embodiment, the test substrate laminate also comprises pads for purging the sample dispenser and cleaning the testing area.

In another embodiment, the test substrate apparatus is used with a blood parameter measurement system in which the system operates automatically to draw blood samples at suitable, programmable frequencies to analyze the drawn blood samples and obtain the desired blood readings such as glucose levels, hematocrit levels, hemoglobin blood oxygen saturation, blood gasses, lactates or any other parameter as would be evident to persons of ordinary skill in the art.

Reference will now be made in detail to specific embodiments of the invention. While the invention will be described in conjunction with specific embodiments, it is not intended to limit the invention to one embodiment. Thus, the present invention is not intended to be limited to the embodiments described, but is to be accorded the broadest scope consistent with the disclosure set forth herein.

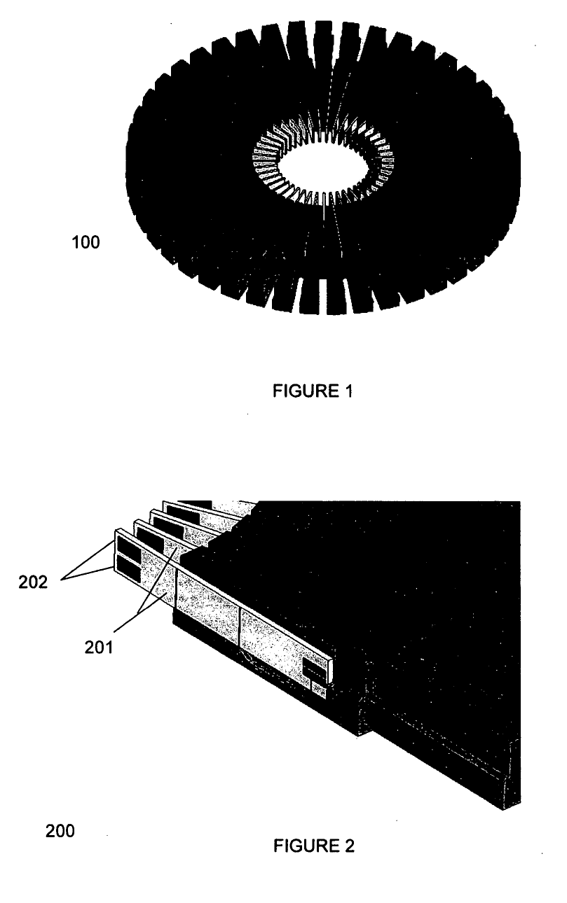

FIG. 1 is a top perspective view of one embodiment of the test substrate holding apparatus of the present invention, wherein the test substrate is held in a carousel-type arrangement. As shown in FIG. 1, carousel 100 preferably stores at least twenty test strips at full capacity. In operation, carousel 100 advances to the next clean test strip when a testing event is initiated or when a used test strip is removed. The carousel 100 comprises a plurality of substantially linear structures 105, referred to as testing strip holders, protruding radially out from a central hub 110.

FIG. 2 is an enlarged cross-sectional view of one embodiment of the carousel test substrate holding apparatus of the present invention 200, as shown in FIG. 1. Referring now to FIG. 2, a plurality of chemical test substrate strips 201 are arranged in the carousel 200 (not shown in its entirety) and, more particularly, physically engaged with testing strip holders 205. Chemical test substrate strips 201 preferably include electrode points 202. Electrode points 202 are positioned to contact the monitor interface and thus transmit information to the host monitor.

In one embodiment, the carousel is used in operation with conventional physiological monitors, such as conventional blood glucose analyzer. The carousel is loaded with conventional testing strips and sealed in a cassette form by the manufacturer. Operationally, the carousel cassette is configured so that a first strip is rotated into measurement position, e.g. where electrodes 202 are in electro-chemical contact with the testing mechanisms of the blood glucose analyzer. A drop of blood is then dispensed on the measurement chemistry of the strip and the analyzer is activated.

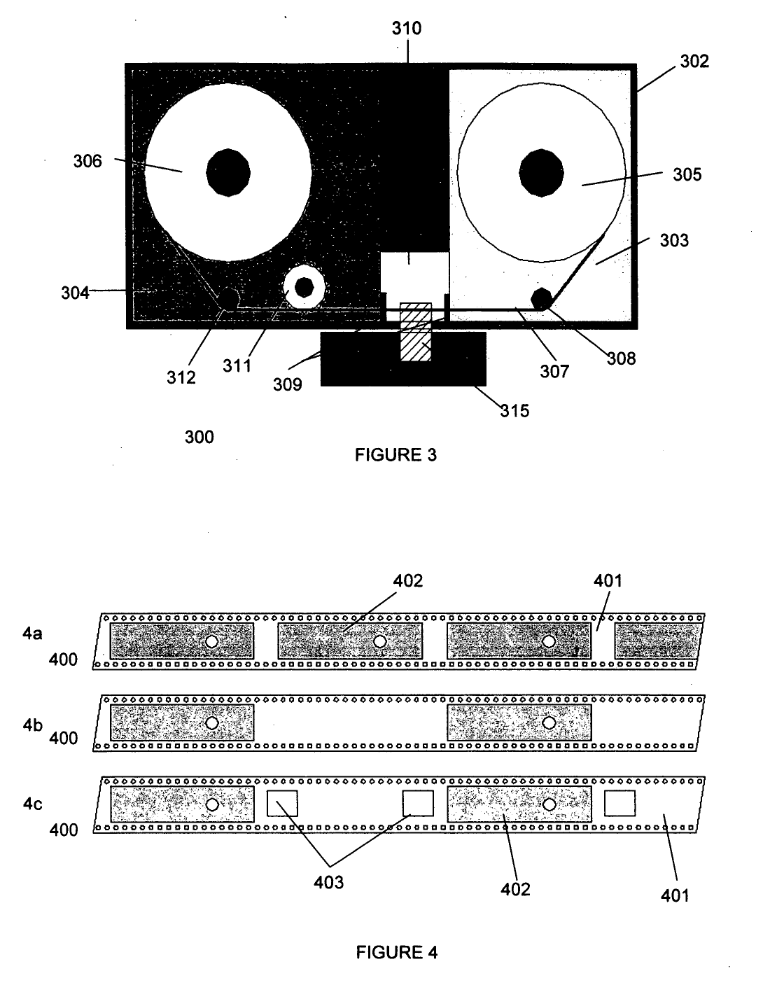

In one embodiment, the test substrate handling device of the present invention is employed for storing test substrates or strips and positioning the substrate for subsequent testing. FIG. 3 is a detailed illustration of one embodiment of a test substrate handling apparatus of the present invention. In one embodiment, test substrate handling apparatus 300 is a cassette. Test substrate handling apparatus 300 comprises outer casing 302, first sealed compartment 303, and second sealed compartment 304. Preferably, first sealed compartment 303 and second sealed compartment 304 are sealed within outer case 302. First sealed compartment 303 is preferably used to store unused test substrates mechanically attached to a transport tape for blood parameter analysis. Second sealed compartment 304 is preferably employed to store used test substrate tape for later disposal.

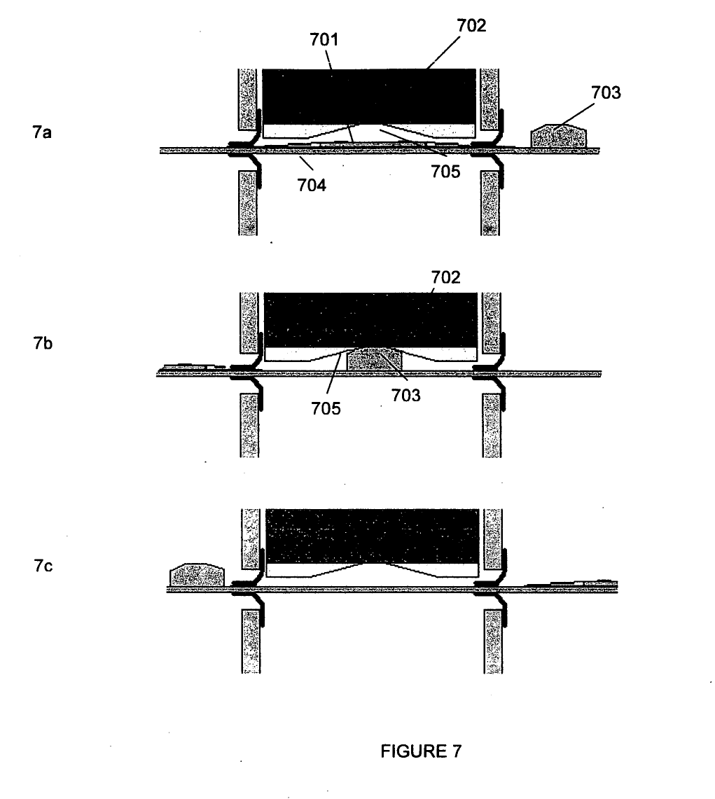

In one embodiment, cassette 300 further comprises a continuous tape laminate 307 containing a plurality of test substrates (not shown), described in greater detail below with respect to FIG. 4. The tape laminate 307 portion which contains unused test substrates is wound on a rotary dispensing drum 305, housed and sealed in first compartment 303. Tape laminate 307 is transported through the cassette via gear 311. Tape laminate 307 is guided from rotary drum 305 towards sample dispenser 310 via first plastic roller 308. First plastic roller 308 preferably contains a groove (not shown) for tape laminate 307 to grip. The test strip (not shown) on tape laminate 307 is positioned for testing at an appropriate position near sample dispenser 310 when a testing event is initiated. Sample dispenser 310 is preferably housed in cassette 300 and is sealed from both new and used test substrates to avoid contamination until a testing event is initiated. The operation of one exemplary use of the test substrate handling mechanism in conjunction with a testing apparatus is described in greater detail below with respect to FIGS. 7a, 7b, and 7c.

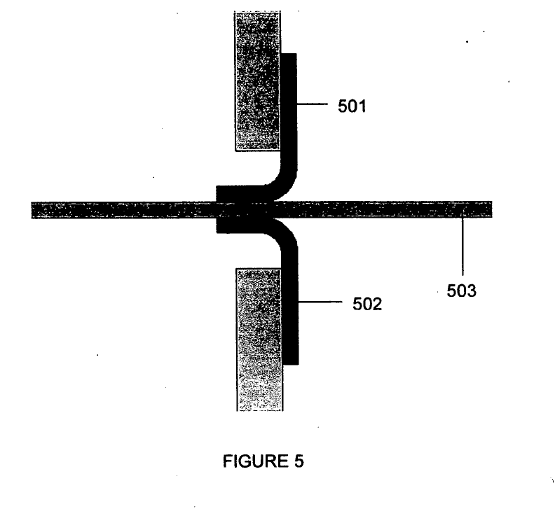

In one embodiment, the test substrate handling device of the present invention is also employed for holding used test substrate elements. In another embodiment, the test substrate handling device of the present invention is used for holding residual test fluid. When a testing event is complete, the tape laminate 307 portion containing used test strips is advanced and subsequently wound into rotary drum 306, housed and sealed in second compartment 304. Second plastic roller 312 is employed to guide tape laminate 307 into the used tape compartment 304 and wind it into rotary drum 306. The reel of test substrate tape laminate 307 preferably extends between first sealed compartment 303 and second sealed compartment 304. In one embodiment, cassette 300 also comprises tape compartment seals 309, described in greater detail below with respect to FIG. 5. Cassette preferably also comprises monitor interface 315, which is sealed as part of the cassette, and may be optical, electrical, or any other suitable monitoring means. The host monitor (not shown) reads the test substrate through the monitor interface.

In one embodiment, cassette 300 further comprises a window (not shown) formed in the top outer surface of the cassette to allow the user to view the test strips stored in the cassette. When the test strips are numbered, the user can determine the number of test strips remaining on rotary drum 305. In the alternative, the visual reading of the number on the strip may be replaced by a suitable electronic circuitry for alerting the user when the number of unused test strips is depleted to a predetermined level.

FIGS. 4a, 4b, and 4c depict three embodiments of the test substrate tape laminate of the present invention. As shown in FIGS. 4a and 4b, test substrate laminate 400 comprises tape 401 and at least one test substrate 402. The test substrate laminate is a continuous tape 401 that holds the test substrate 402 in such an arrangement that it can be presented to the sample dispenser (not shown) for blood parameter analysis. FIGS. 4a and 4b depict different spacing arrangements for the at least one test substrate 402 on the tape laminate 401. The test substrates 402 are spaced such that the next new test substrate is sealed in the unused test substrate compartment and the used substrate is sealed in the used test substrate compartment.

As shown in FIG. 4c, in one embodiment, test substrate tape laminate further comprises pads 403 for purging the sample dispenser. Pads 403 may be cleaning pads, wick pads, or any other suitable pad as are known to those of ordinary skill in the art. The use of the pad is described in greater detail below with respect to FIGS. 7a, 7b, and 7c.

FIG. 5 is an illustration of one embodiment of the tape compartment seal of the test substrate handling apparatus of the present invention. Referring back to FIG. 3, cassette 300 further comprises tape compartment seals 309 to provide a barrier or seal or tortuous path between adjacent compartments and allow the tape laminate to pass from one compartment to the other.

Now referring to FIG. 5, one embodiment of a tape compartment seal is depicted. Tape compartment seal comprises first seal wipers 501 and second seal wipers 502, located above and below the travel plane of the test substrate tape laminate 503, respectively. Seal wipers 501, 502 are flexible and conform to the shape of test substrate tape laminate 503. The seal allows the tape laminate containing a test substrate to move in only one direction, towards the sample dispenser compartment, and subsequently to the used tape compartment. Thus, the tape compartment seal isolates the tape compartment from particles, air, dust and fluid that may be present in the adjacent compartment.

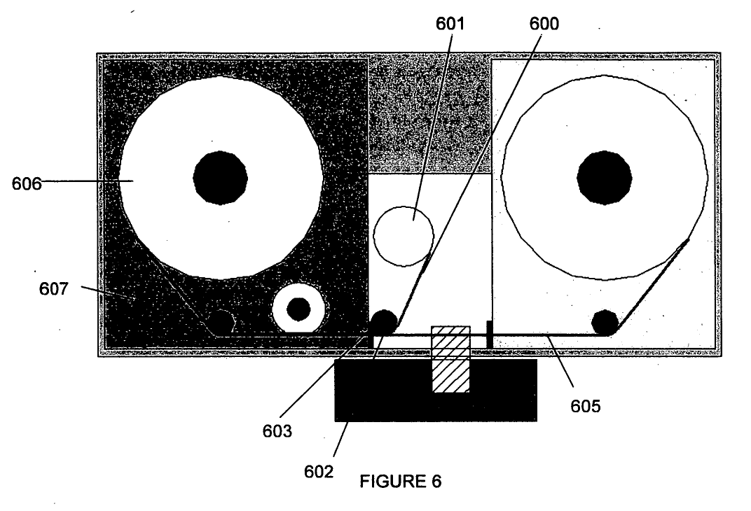

FIG. 6 depicts one embodiment of a cover layer for the test substrate laminate tape of the present invention, shown in FIG. 5. Cover layer 600 originates from cover layer laminate roll 601. In one embodiment, cover layer laminate roll 601 is housed in the same chamber as the sample dispenser shown in FIG. 3. Once test substrate laminate tape 605, containing an area with a test strip (not shown), is transported to the sample dispenser area and is used, it is laminated via cover laminate roll 601. Cover laminate roll 601 is guided by third plastic roller 602. The covered test substrate strip is subsequently transported through the second tape compartment seal 603, thus reducing the possible contamination across seal barriers, by covering the used test strip. The dual layer tape is then wound into rotary drum 606, housed in second compartment 607.

FIGS. 7a, 7b, and 7c illustrate the operational steps of one embodiment of the test substrate handling apparatus further comprising a cleaning pad, used in conjunction with a fluid dispensing system for fluid test samples.

In one embodiment, as shown in FIG. 7a, the chemical test substrate handling apparatus is used in an automated blood parameter analysis and measurement system. In operation, once the test substrate 701 reaches sample dispenser 702, a blood sample is withdrawn from a vascular access point. In one embodiment, sample dispenser 702 is a dispensing valve. The sample is positioned on the test substrate 701 for optimal analysis by the monitor interface (not shown) and host monitor (not shown). Once a sample is taken and deposited on the test substrate, tape laminate 704, which contains test substrate 701, is advanced.

Pad 703 is optionally provided on the tape laminate and positioned in between each test substrate, as shown in FIG. 4c, for purging the sample dispenser once sample collection and analysis of the blood sample is complete. Pad 703 may be a wick pad or cleaning pad of appropriate means to clean the sample dispenser.

If pad 703 is present, after sample collection, the tape laminate 704 is advanced to the next wick pad 703 and positioned at the mouth 705 of sample dispenser 702, as shown in FIG. 7b. Wick pad 703 is then used to clean the sample dispenser 702. The prior sampling and testing by the fluid access system generally leaves residual fluid in and around the dispensing valve 705. When the wick pad 703 comes into contact with dispensing valve 705, the wick pad 703 absorbs the residual fluid from dispensing valve 705. When the cleaning of the dispensing valve 705 is complete, dispensing valve 705 is closed and tape laminate 704 is advanced forward, as shown in FIG. 7c. In one embodiment, both the wick pads and test substrates are advanced and isolated to prevent contamination. In another embodiment both the wick pads and test substrates are sealed with a laminate tape to prevent further contamination.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention is not to be limited to the details given herein, but may be modified within the scope of the appended claims.

We claim:

1. An apparatus for holding and positioning a plurality of test substrates for analysis and measurement of blood parameters, comprising:
a first storage means for storing unused test substrate;
a means for advancing and individually positioning at least one unused test substrate for receiving a sample from a sample dispenser; and
a second storage means for storing used test substrate.

2. The apparatus of claim 1 wherein said apparatus comprises a cassette.

3. The apparatus of claim 1 wherein said apparatus comprises a strip handling carousel.

4. The apparatus of claim 2 wherein said first storage means and said second storage means is a sealed compartment within the cassette.

5. The apparatus of claim 1 wherein said test substrate further comprises a continuous tape laminate.

6. The apparatus of claim 5 wherein said tape laminate further comprises a pad.

7. The apparatus of claim 6 wherein said pad is a wick pad.

8. The apparatus of claim 6 wherein said pad is a cleaning pad.

9. An apparatus for holding and positioning a plurality of test substrates for analysis and measurement of blood parameters, comprising a cassette, wherein said cassette further comprises:
an outer case;
a first sealed compartment for storing a plurality of unused test substrates;
a second sealed compartment, for storing a plurality of used test substrates;
a test substrate pathway extending between said first sealed compartment and said second sealed compartment
a sample dispenser in fluid communication with said pathway; and
a monitor interface.

10. The apparatus of claim 9 wherein the cassette further comprises a tape laminate, wherein said tape laminate is used to hold the test substrate.

11. The apparatus of claim 10 wherein said tape laminate further comprises a pad.

12. The apparatus of claim 11 wherein said pad is a wick pad.

13. The apparatus of claim 11 wherein said pad is a cleaning pad.

14. The apparatus of claim 9 wherein the cassette further comprises a laminate tape compartment seal.

15. The apparatus of claim 9 wherein the cassette further comprises at least one plastic roller for guiding the test substrate through the cassette.

16. The apparatus of claim 9 wherein the cassette further comprises a gear for advancing the test substrate through the cassette.

17. The apparatus of claim 9 wherein the cassette further comprises a tape laminate cover roll.
